(19) United States
(12) Patent Application Publication
(10) Pub. No.: US 2012/0145156 A1
(43) Pub. Date: Jun. 14, 2012

(54) PHONATION ASSISTANCE DEVICE FOR A TRACHEOTOMY PATIENT

(75) Inventors: Frederic Lofaso, Clamart (FR); Helene Prigent, Paris (FR); Karl Leroux, Touquin (le Tau) (FR)

(73) Assignee: ASSISTANCE PUBLIQUE - HOPITAUX DE PARIS, Paris (FR)

(21) Appl. No.: 13/319,273

(22) PCT Filed: May 5, 2010
(86) PCT No.: PCT/FR2010/050858

§ 371 Date: Feb. 29, 2012

(30) Foreign Application Priority Data
May 7, 2009 (FR) 0953058

(51) Int. Cl. A61M 16/20

(52) U.S. Cl. 128/205.24; 128/207.16

(57) ABSTRACT

The invention relates to a phonation assistance device for a tracheotomy patient, including: an exhalation circuit connected to a tracheotomy cannula inserted in the trachea of the patient, the exhalation circuit including an outlet opening for the passage of the air exhaled by the patient; and a valve for opening/closing the outlet opening for normally assuming, when the patient inhales, a position for closing the outlet opening and, when the patient exhales, a position for closing the outlet opening. It also includes means for the positive priority control of the valve for selectively moving the valve into the closed position when the patient exhales.

### CROSS REFERENCE TO PRIOR APPLICATIONS

This application is a U.S. National Phase application under 35 U.S.C. §371 of International Application No. PCT/FR2010/050858, filed on May 5, 2010 and claims benefit of priority to French Patent Application No. 0953058, filed on May 7, 2009. The International Application was published in French on Nov. 11, 2010 as WO 2010/128250 A1 under PCT Article 21 (2). All of these applications are herein incorporated by reference.

### TECHNICAL FIELD

The present invention relates to a phonation assistance device for a tracheotomy patient, of the type comprising:
an exhalation circuit connected to a tracheotomy cannula inserted in the trachea of the patient, the exhalation circuit including an outlet opening for the passage of the air exhaled by the patient; and
a valve for opening/closing the outlet opening for normally assuming, when the patient inhales, a position for closing the outlet opening and, when the patient exhales, a position for opening the outlet opening.

### BACKGROUND

In certain medical situations where a patient encounters difficulties in breathing, it is necessary to perform a tracheotomy, which is an operation in which an incision is made in the patient's neck so as to establish communication with the inside of the trachea.

A cannula, called tracheotomy cannula and through which outside air can penetrate, is then inserted through the incision in the trachea, thereby making it possible to ensure spontaneous or mechanical pulmonary ventilation of the patient without going through the upper respiratory paths.

However, the implantation of a tracheotomy cannula in a patient's trachea generally does not allow the passage of the exhaled air toward the upper respiratory paths, which are responsible for ensuring the operation of the vocal cords to allow the patient to produce phonemes.

Devices of the aforementioned type are known making it possible to mechanically ventilate the patient using a ventilator while allowing him to keep the possibility of producing phonemes. In such devices, a check valve is positioned on the tracheotomy cannula, allowing the inhaled flow of aft to penetrate the trachea through that valve, while the exhaled air can only exit toward the patient's vocal cords if there is sufficient space between the cannula and the trachea, thereby allowing him to preserve the use of speech.

However, as long as the check valve is present on the tracheotomy cannula, the patient is required to breathe through his mouth, leading to dehydration of the respiratory paths. Furthermore, in most cases, the valve cannot be removed by the patient alone, and removing the valve requires disconnecting, then reconnecting the ventilator, which can be dangerous for the patient, in particular when the patient is at home and not in a medical setting. Furthermore, if the exhalation through the mouth is not complete due to an excessive resistance between the cannula and the trachea and/or an overly short exhalation time imposed by the ventilator, there is a risk of pulmonary hyperinflation.

The invention aims to propose a simple device that makes it possible to facilitate the breathing and speech of a patient having undergone a tracheotomy, while preventing or at least reducing both the dehydration of the patient's respiratory paths and the risk of pulmonary hyperinflation.

### SUMMARY

To that end, the invention relates to a device of the aforementioned type, characterized in that it also comprises priority positive control means of the valve, adapted to selectively bring the valve into the closing position when the patient exhales.

The device according to the invention can include one or more of the following features:
the priority positive control means comprises a first solenoid valve for controlling the valve, and a control switch for the first solenoid valve;
the valve comprises a housing in which the exhalation circuit emerges and comprising an air outlet opening, and a member steered by the pressure housed in the housing and adapted to assume a closed position in which it closes the outlet opening, and an open position in which it opens the outlet opening;
the priority positive control means comprises a pressure generator selectively connected, via the first solenoid valve, to the member controlled by the pressure;
the pressure generator is a continuous fan turbine;
the device comprises an inhalation circuit permanently connected to the cannula and comprising an inlet opening allowing the passage of air inhaled by the patient;
the device comprises a ventilator whereof the discharge is connected to the inhalation circuit;
the ventilator comprises a second solenoid valve and is selectively connected to the valve via the second solenoid valve;
the first solenoid valve is selectively controlled by the switch and b the ventilator; and
the device comprises a tracheotomy cannula intended to be inserted into the trachea of the patient and connected to the exhalation circuit.

BRIEF DESCRIPTION OF THE DRAWINGS

The invention will be better understood upon reading the following description, provided solely as an example and done in reference to the appended drawings, in which:

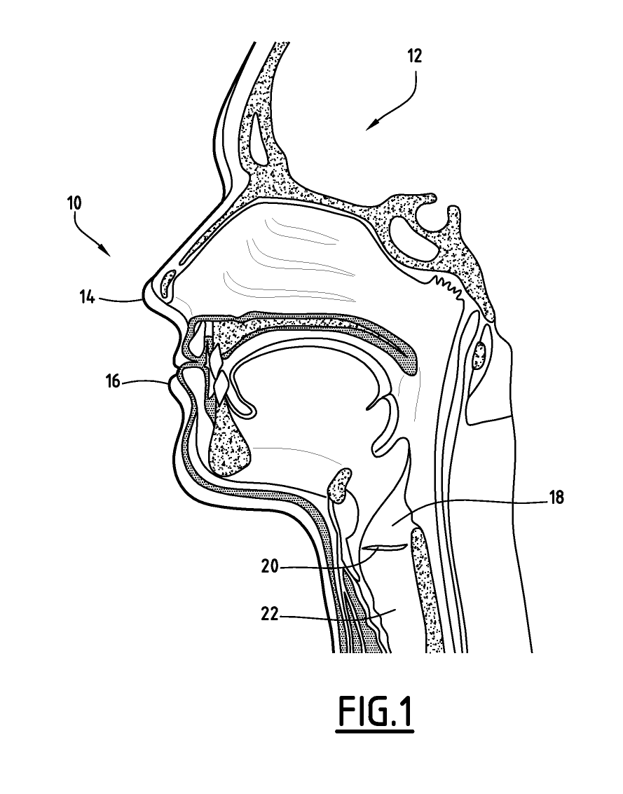

FIG. 1 is a cross-sectional profile view of the upper respiratory paths of a human being;

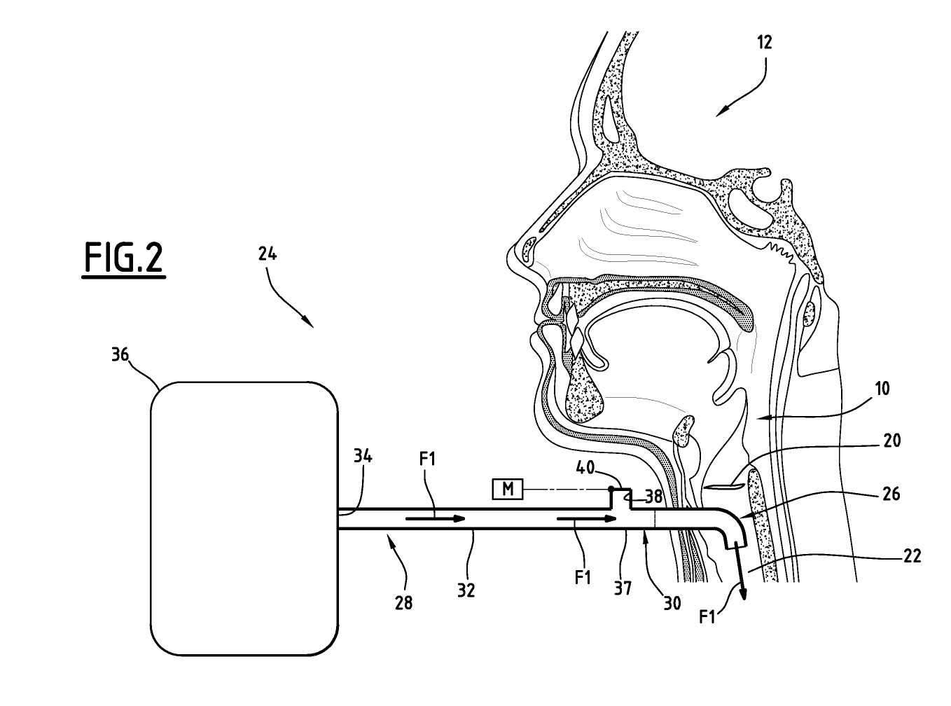

FIG. 2 is a diagrammatic view illustrating the operating principle of a device according to a first embodiment of the invention during an inhalation phase;

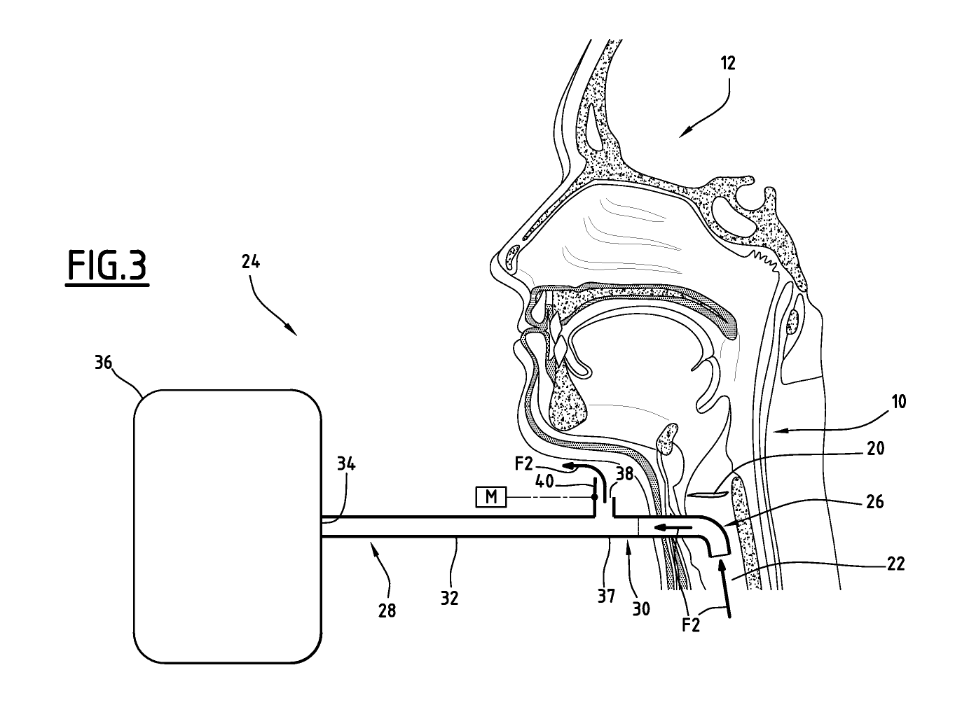

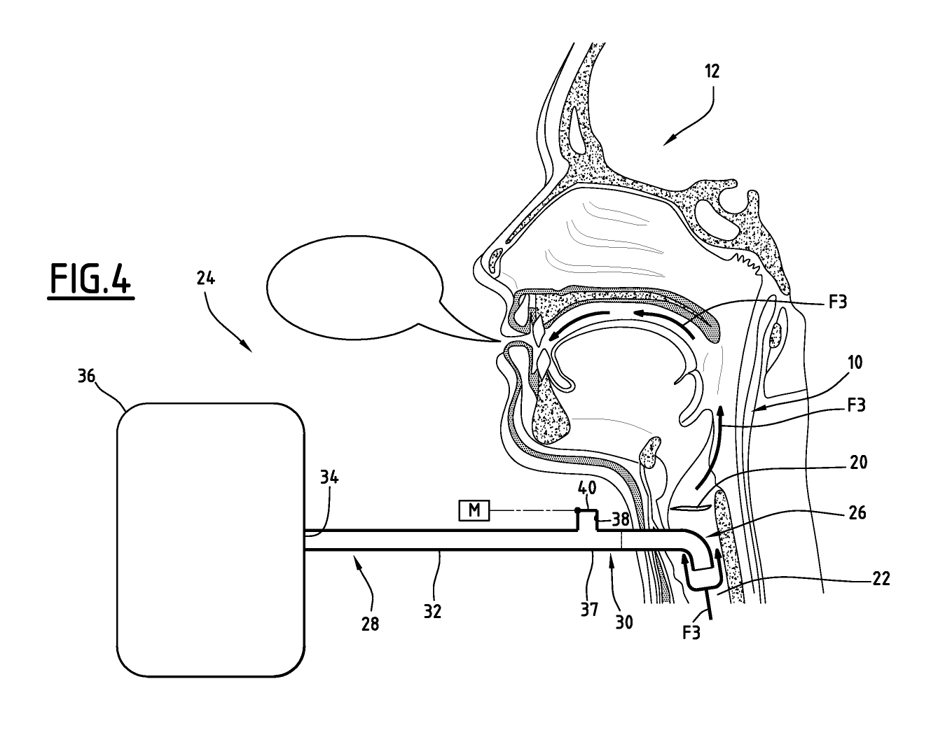

FIGS. 3 and 4 are views similar to that of FIG. 2 during first and second exhalation phases, respectively;

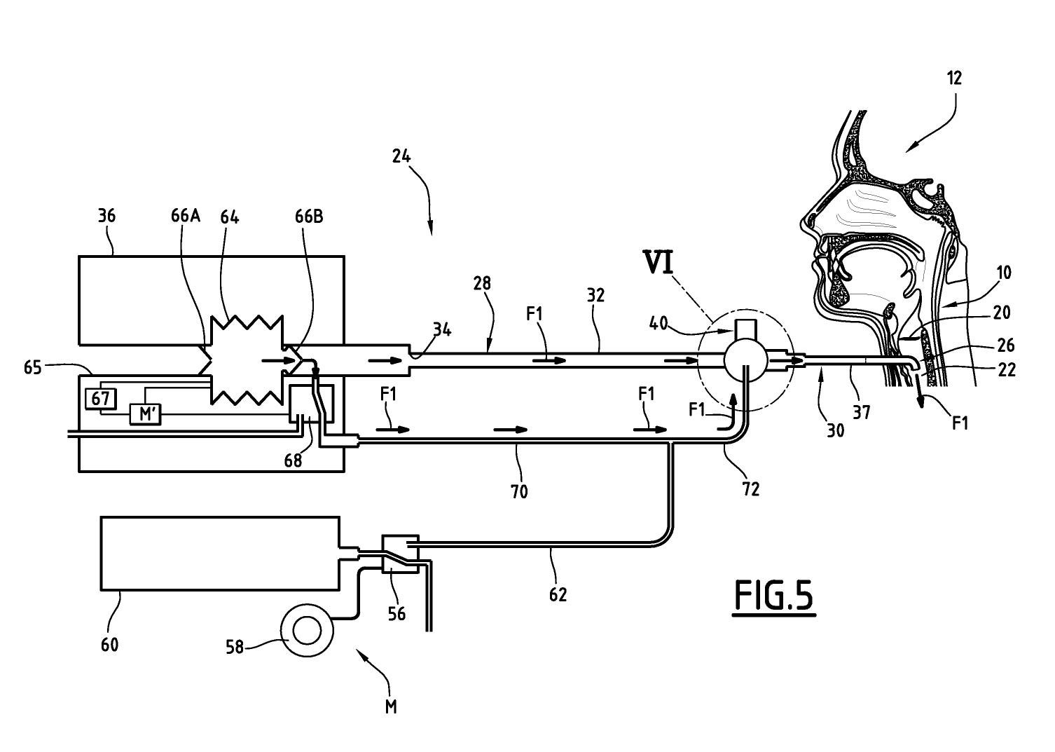

FIG. 5 is a view diagrammatically illustrating the device of FIG. 2 during a first inhalation phase;

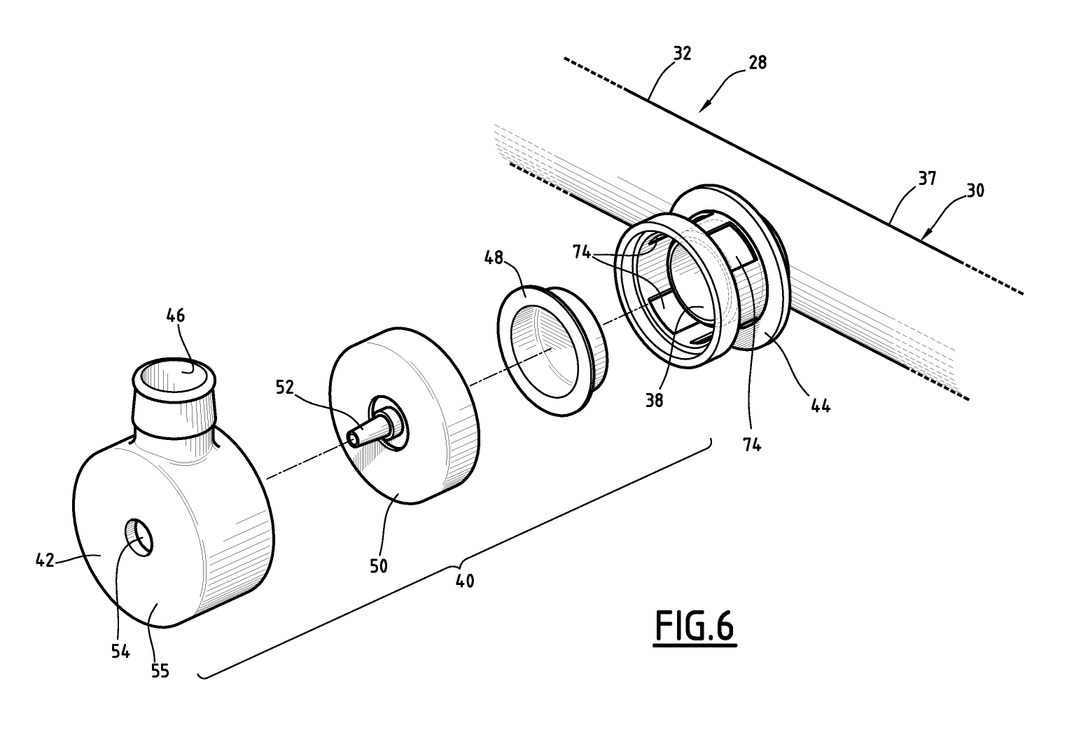

FIG. 6 is a perspective view of the valve of the device of FIG. 5;

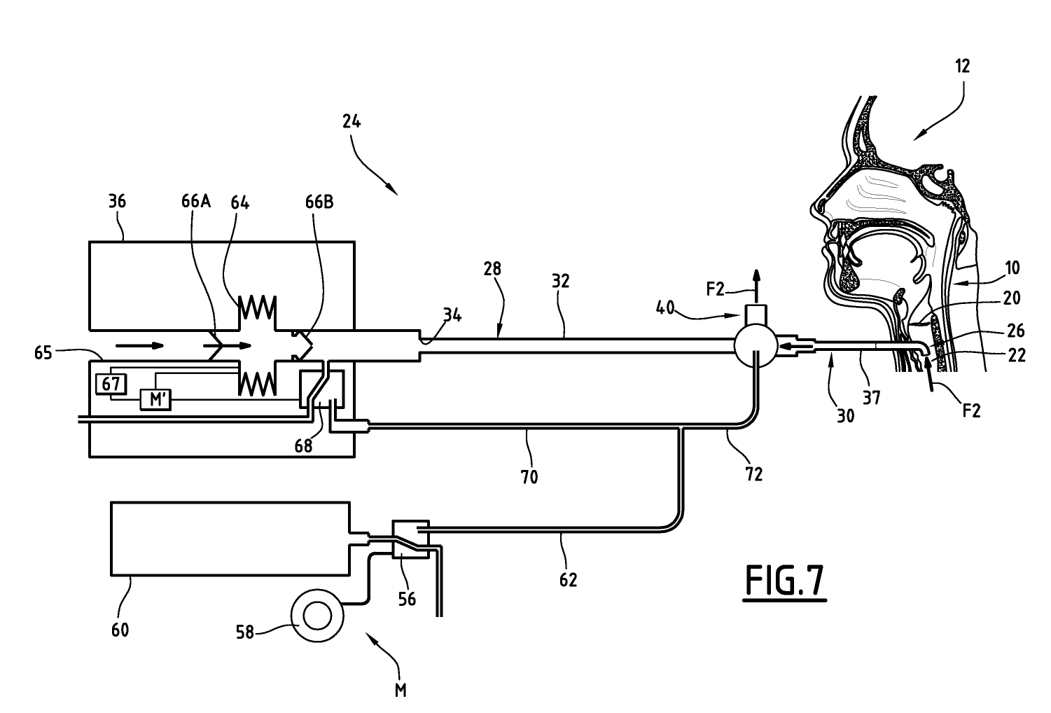

FIGS. 7, 8 and 9 are views similar to that of FIG. 5 during a first exhalation phase, a second inhalation phase, and a second exhalation phase, respectively; and

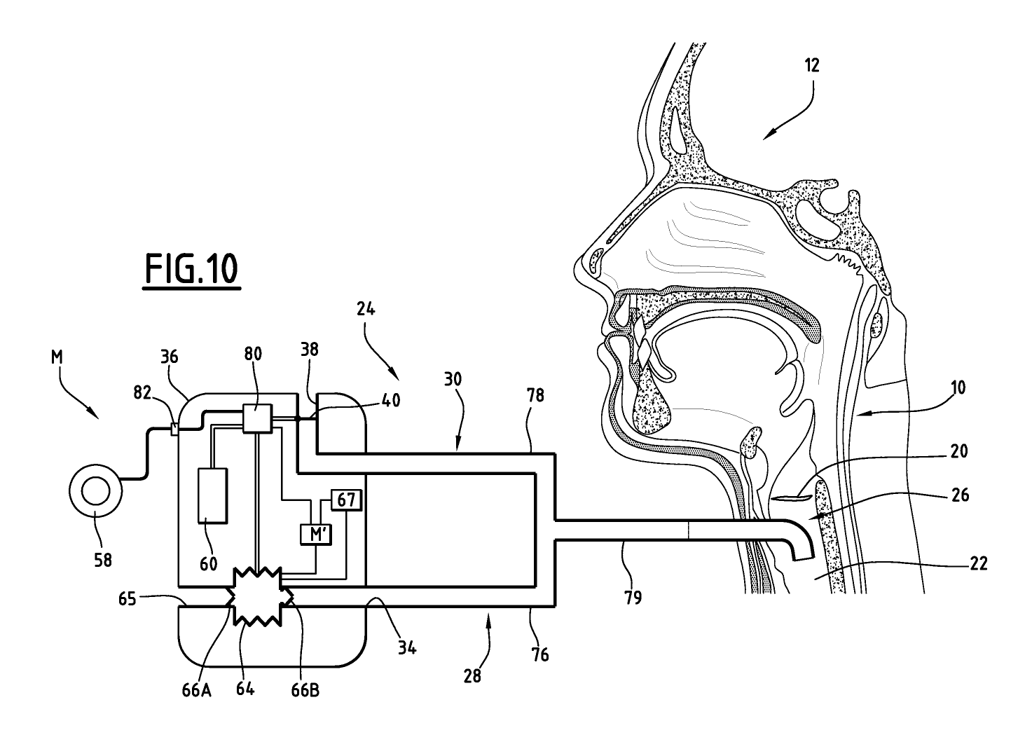

FIGS. 10, 11, 12 and 13 are views diagrammatically illustrating a device according to second, third, fourth, and fifth embodiments of the invention, respectively.

### DETAILED DESCRIPTION

In order to better understand the following description, several reminders about human anatomy are provided in reference to FIG. 1, in which the upper respiratory paths 10 of a human being 12 are shown.

The respiratory paths, also called airways, are channels that allow air to pass from the nose 14 and the mouth 16 to the lungs and alveoli during ventilation, also called respiration. The term upper respiratory paths 10 designates the part of the respiratory paths situated above the larynx 18, and lower respiratory paths designates the part of the respiratory paths situated below the larynx 18.

The larynx 18 is an organ situated at the throat and containing the vocal cords 20, the vibration of which allows phonation, i.e. the production of vocal sounds. The larynx 18 is extended toward the lower respiratory paths by the trachea 22.

The trachea 22 is a conduction zone making it possible, during inhalation, to convey air from the larynx 18 into the bronchi, and to make the carbon dioxide-rich air exit during exhalation.

When a patient needs continuous mechanical ventilation, which makes it possible to supply spontaneous ventilation using a device called a ventilator, or respirator, a tracheotomy may be necessary to ensure the most reliable and effective interface possible between the ventilator and the patient.

FIG. 2 diagrammatically illustrates the operating principle of a device 24 according to a first embodiment of the invention, and which is a phonation assistance device for a patient 12 having undergone a tracheotomy.

The device 24 comprises a tracheotomy cannula 26 inserted into the trachea 22 of a patient 12, an inhalation circuit 28 and an exhalation circuit 30 connected to the cannula 26.

The inhalation circuit 28 is formed by a tube 32 continuously connected to the cannula 26 and including an inlet opening 34 allowing the passage of air inhaled by the patient 12.

The device 24 also comprises a ventilator 36 whereof the discharge is connected to the inlet opening 34 of the inhalation circuit 28. The ventilator 36 operates discontinuously, so as to generate a flow of air only during the inhalation phases.

The inhalation circuit 28 is thus delimited on one side by the ventilator 36, and on the other side by the cannula 26.

The exhalation circuit 30 is formed by a distal segment 37 of the tube 32 and comprises a tubular outlet 38 injected on the tube 32, allowing the passage of air exhaled by the patient 12, and an opening/closing valve 40 of the outlet opening 38.

The exhalation circuit 30 is thus delimited on one side by the bleed of the outlet opening 38 on the tube 32, and on the other side by the cannula 26.

The inhalation circuit 28 is therefore formed by the entire tube 32 while the exhalation it 30 is formed by part of the tube 32.

As will be detailed later, the valve 40 can be moved under the effect of the patient's breathing, between a closed position of the outlet opening 38 when the patient 12 inhales, and an open position of the outlet opening 38 when the patient 12 exhales.

As illustrated in FIG. 2, during the inhalation phase, the valve 40 is in the closed position of the outlet opening 38.

The ventilator 36 then delivers a flow of air, shown by the arrows F1, which penetrates through the inlet opening 34 and inside the inhalation circuit 28, as far as into the cannula 26 to emerge in the trachea 22 of the patient 12 toward the latter's lungs.

In reference to FIG. 3, during the exhalation phase, the valve 40 is in the open position of the outlet opening 38.

The air previously inhaled is then exhaled (arrows F2) and rises in the trachea 22 toward the cannula 26. The exhaled flow of air F2 next penetrates the cannula 26 as far as the exhalation circuit 30 to emerge outside the device 24 through the outlet opening 38.

During this exhalation phase, the exhaled flow of air F2 passing in the cannula 26, the patient 12 does not have the ability to speak.

In order to allow him to express himself when he wishes, the device 24 comprises a means M for controlling the valve 40 that can be actuated by the patient 12, as will be explained in more detail later, to selectively bring the valve 40 into the closed position of the outlet opening 38 when he exhales.

As shown in FIG. 4, when the patient 12 actuates the control means M, the valve 40 is closed and blocks the exhaled air. All of the exhaled air is then evacuated through the upper respiratory paths 10. The patient 12 can thus use this flow of air F3 to vibrate his vocal cords 20 and produce phonemes.

FIG. 5 diagrammatically illustrates the first embodiment of the device 24.

In reference to FIG. 6, the valve 40 comprises a housing 42 in which the exhalation circuit 30 emerges through the outlet opening 38.

The housing 42 is mounted on the end 44 of the exhalation circuit 30, forming a plug covering that end 44, and comprises a side air outlet opening 46.

The valve 40 also comprises a membrane 48 housed in the housing 42 and positioned on the outlet opening 38, and a tip 50 housed in the housing 42 and positioned on the membrane 48.

The membrane 48 is made from a flexible plastic material so as to assume, under the effect of an outside overpressure, a closed position of the outlet opening 38, and without pressure or with an internal overpressure, an open position of the outlet opening 38.

The tip 50 comprises a connecting member 52 protruding toward the outside of the housing 42 through a hole 54 formed in a bottom wall 55 of the housing 42. The connecting member 52 is connected to a pressure source, as will be explained in more detail hereafter.

As illustrated in FIG. 5, the means M for controlling the valve 40 comprises a first solenoid valve 56 for controlling the valve 40, a switch 58 for controlling the first solenoid valve 56, and a pressure generator 60.

The pressure generator 60 is selectively connected, via the first solenoid valve 56, to the outer surface of the membrane 48 of the valve 40 by a tube 62 connected to the member 52 (FIG. 6) of the tip 50.

In the considered example, the pressure generator 60 is formed by a continuous fan turbine, delivering an overpressure from 4 to 20 mbar.

The ventilator 36 comprises a bellows 64 controlled by a motor 67 and an air intake conduit 65 connected to the bellows 64 and emerging on the outside of the ventilator 36. The discharge of the bellows 64 is connected to the inlet opening 34 of the inhalation circuit 28.

The ventilator 36 also comprises two check valves 66, a first valve 66A being positioned in the conduit 65, allowing the entry of outside air into the bellows 64 and preventing the air confined in the bellows 64 from exiting outside the ventilator 36. The second valve 66B is positioned in the discharge of the bellows 64, allowing the air confined in the bellows 64 to penetrate the inhalation circuit 28 and preventing that air from again penetrating the bellows 64.

The ventilator 36 also comprises a second solenoid valve 68 and a means M′ for controlling the second solenoid valve 68. The control means M′ makes it possible, through the second solenoid valve 68, to selectively connect the bellows 64 to the outer surface of the membrane 48 of the valve 40 by a tube 70 connected to the tube 62 and therefore to the member 52 (FIG. 6) of the tip 50.

The tubes 62 and 70 thus come together to form a single branch 72 connected to the tip 50.

The control means M′ is also connected to the motor 67 of the bellows 64 so as to synchronize the blowing with the opening of the second solenoid valve 68 on the tube 70 and therefore with the closing of the valve 40.

During a first inhalation phase shown in FIG. 5, the switch 58 is not actuated so that the pressure generator 60 is not connected to the valve 40, while the means M′ automatically control the second solenoid valve 68 so as to connect the bellows 64 to the valve 40, the bellows 64 initially containing a volume of air.

To generate blowing during the inhalation, the bellows 64 then delivers a flow of air F1, part of which passes through the tubes 70, 72 to penetrate inside the housing 42 of the valve 40 through the tip 50. This flow of air F1 generates a pressure that presses the membrane 48 against the outlet opening 38, covering the latter sealably.

The other part of the flow of air F1 passes along the inhalation circuit 28 as far as the tracheotomy cannula 26 to emerge in the trachea 22 of the patient 12 as far as into the latter's lower respiratory paths.

At the end of this first inhalation phase, the bellows 64 is emptied of all or some of its air, and its volume is reduced.

During a first exhalation phase illustrated in FIG. 7, the switch 58 is still not actuated, so that the pressure generator 60 is still not connected to the valve 40, while the means M′ automatically control the second solenoid valve 68 so as to no longer connect the bellows 64 to the valve 40.

No more pressure is then applied on the membrane 48, which thus opens the outlet opening 38.

The flow of air F2 exhaled by the patient 12 rises through the trachea 22, penetrates the cannula 26, and emerges toward the outside through the outlet opening 38 and the outlet opening 46 of the housing 42.

Windows 74 (FIG. 6) are formed at the end 44 of the exhalation circuit 30, around the outlet opening 38, so as to allow the exit of the exhaled air flow F2.

During this first exhalation phase, the outside air is “aspirated” in the air intake conduit 65 toward the bellows 64, which “inflates” again using the motor 67.

During this first exhalation phase, the patient 12 cannot speak.

When the patient 12 wishes to express himself, he actuates the switch 58, which allows the first solenoid valve 56 to connect the pressure generator 60 to the valve 40.

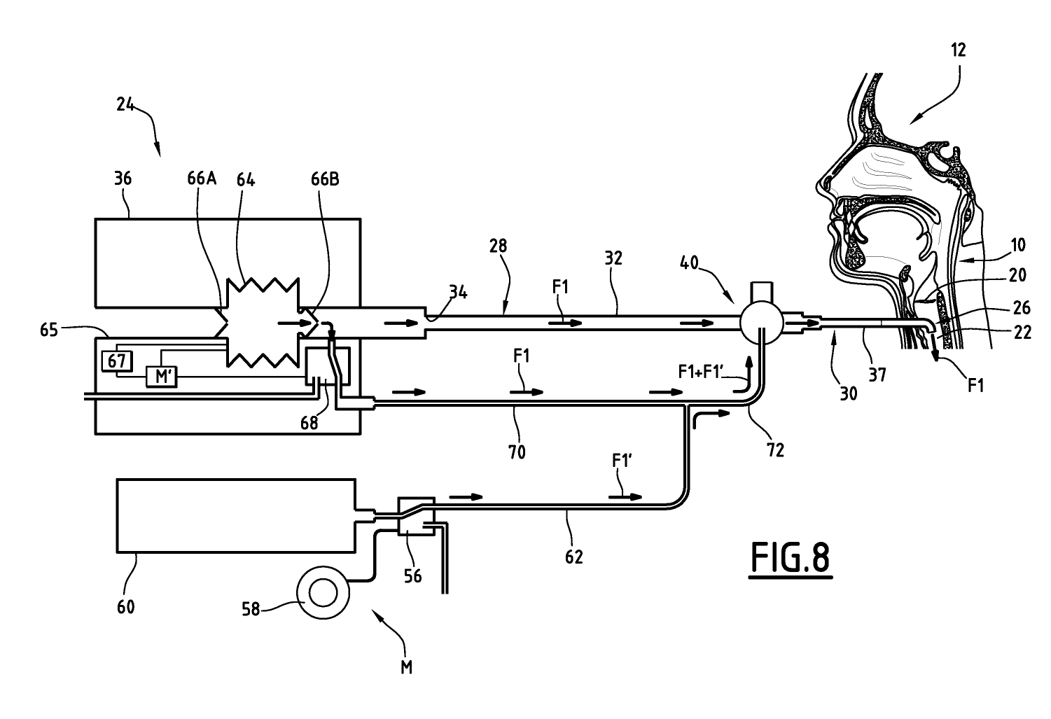

Thus, during a second inhalation phase shown in FIG. 8, the pressure generator 60 delivers a flow of air F1′ that is added to the flow of air F1 delivered by the ventilator 36 so as to cover the outlet opening 38 while allowing the flow of air F1 to circulate through the inhalation circuit 28 as far as the trachea 22 of the patient 12.

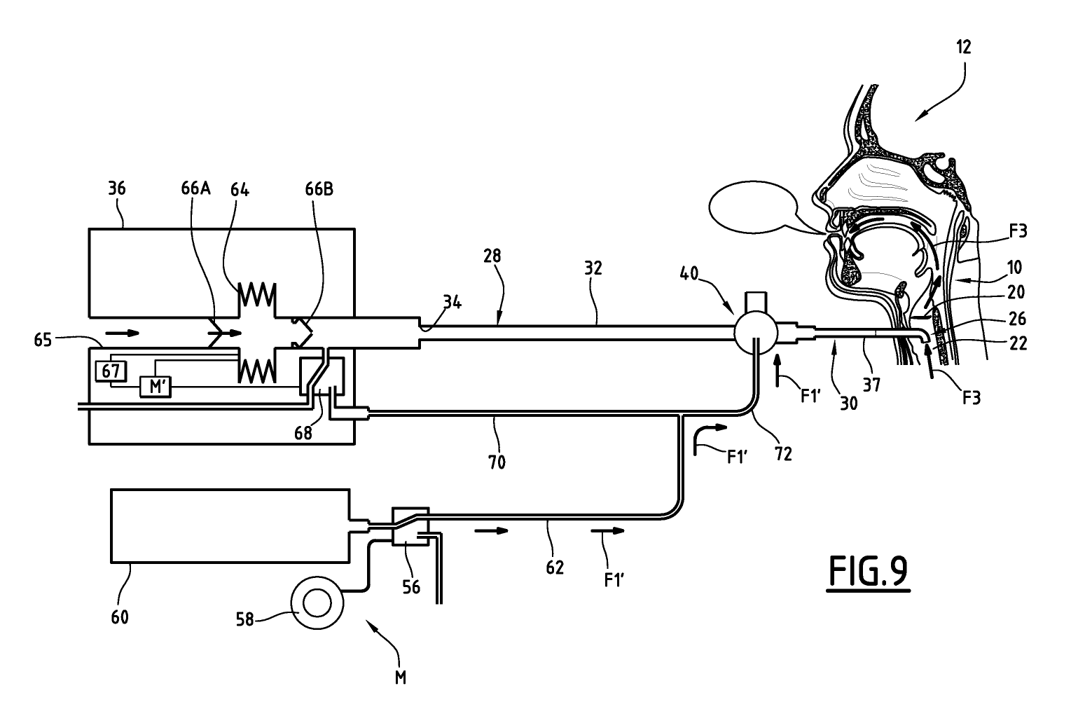

During a second exhalation phase illustrated in FIG. 9, the switch 58 is still actuated so as to close the outlet opening 38 by the flow of air F1′, thereby forming an obstacle to the passage of the flow of air F3 exhaled by the patient 12 through the exhalation circuit 30.

The patient 12 can thus use the exhaled flow of air F3, which escapes exclusively between the trachea 22 and the tracheotomy cannula 26, so as to vibrate his vocal cords 20 and therefore speak.

By closing the exhalation circuit 30, the exhalation can only be done through the upper respiratory paths, and the patient can regain a normal exhalation phonation.

If the patient 12 no longer wishes to speak or if he feels a pulmonary hyperinflation, he need only release the switch 58.

According to a second embodiment of the invention shown in FIG. 10, the inhalation 28 and exhalation 30 circuits are separate from one another and are each formed by a respective tube 76, 78.

The tubes 76, 78 come together to form a single tube 79 connected to the tracheotomy cannula 26.

The end portion of the exhalation circuit 30 comprising the valve 40 and the pressure generator 60 are integrated inside the ventilator 36.

A single solenoid valve 80 connected to the valve 40 is incorporated in the ventilator 36 so as to be under the dual control of the ventilator 36 by the control means M′, and the patient 12 by the switch 58. To that end, the solenoid valve 80 is connected selectively to the bellows 64 by the control means M′ and to the pressure generator 60 by the switch 58, for example using a jack 82.

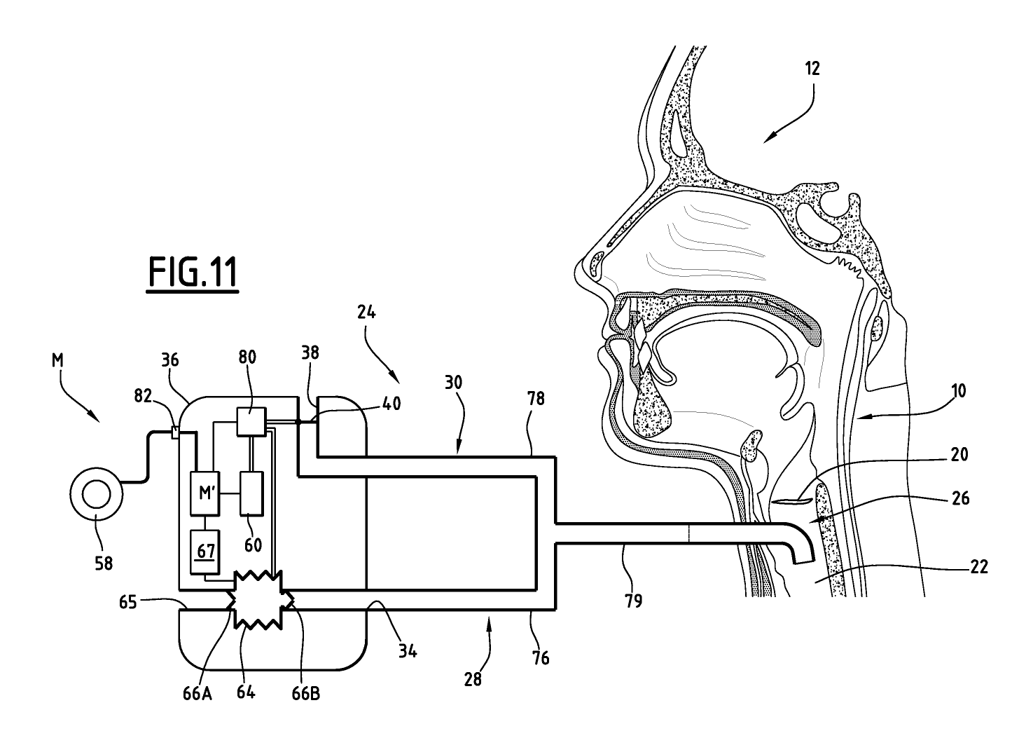

A third embodiment of the invention is illustrated in FIG. 11 and differs from the second embodiment of FIG. 10 in that the solenoid valve 80 is under the control of the ventilator 36 by the control means M′, which in turn are under the control of the patient 12 by the switch 58.

The control means M′ generally comprise a CPU (Central Processing Unit) card, flow rate and pressure sensors, and a control card of the motor 67.

The switch 58 can assume any form adapted to the patient 12, for example a push button or a manual contactor adapted for patients having a motor handicap.

The valve 40 previously described comprises a membrane 48, but it is entirely possible to replace the membrane with a cuff adapted to inflate/deflate under the effect of pressure so as to cover/free the outlet opening 38.

Alternatively, the valve 40 is formed by a non-pneumatic electromechanical system adapted to close/free the outlet opening 38. In that case, the solenoid valve 80 is eliminated and the electromechanical system is directly connected to the control means M′.

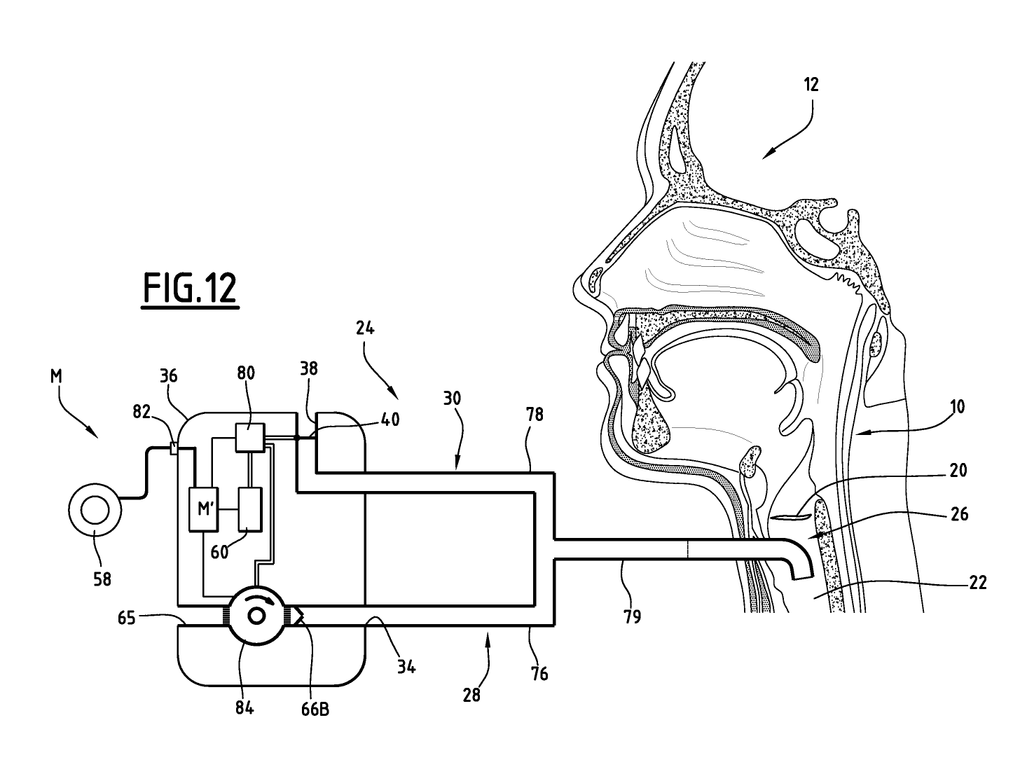

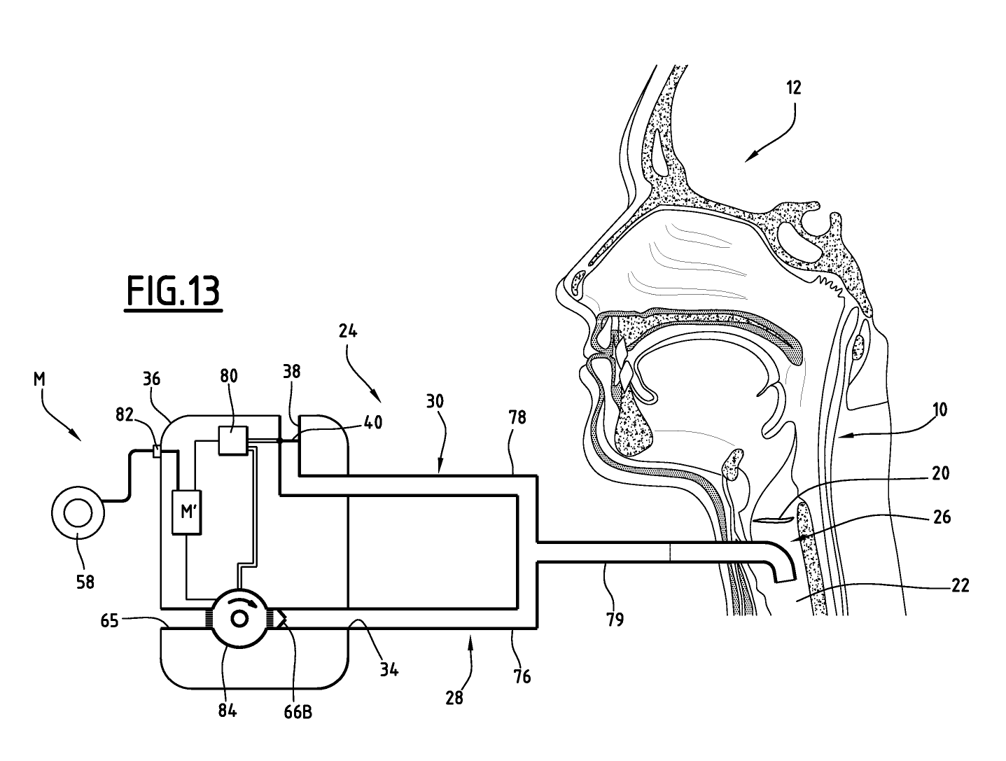

The ventilator 36 can be of any type, for example a type operating with a turbine in place of the bellows 64 and the motor 67, as shown in FIGS. 12 and 13.

FIG. 12 illustrates a fourth embodiment of the invention that differs from the third embodiment of FIG. 11 in that the bellows 64 and the motor 67 are replaced by a continuous turbine 84.

In that case, the valve 66A is also eliminated.

During the inhalation phase, the turbine 84 delivers a flow of air to the patient 12 with a pressure Pi at the outlet of the turbine 84, while the means M′ automatically control the solenoid valve 80 so as to connect the turbine 84 to the valve 40 and thereby cover the outlet opening 38 with a pressure corresponding to Pi.

During the exhalation phase, the turbine 84 still delivers a flow of air, but that flow of air is deflected relative to the inhalation circuit 28 by bypass means (not shown) of the turbine 84. The inhalation circuit 28 is therefore no longer supplied.

Still during the exhalation phase, the solenoid valve 80, under the control of the control means M′, allows the pressure generator 60 to power the valve 40 at a predetermined pressure Pe that can vary from 0 to a value equal to or greater than Pi. In this way, the valve 40 can only open and free the outlet opening 38 when the patient 12 creates an overpressure in the exhalation circuit 30 greater than Pe.

As long as the pressure generated by the patient 12 in the exhalation circuit 30 is below Pe, the air exhaled by the patient 12 cannot emerge toward the outside through the outlet opening 38 and the patient 12 can use all of the exhaled air to speak.

When the pressure generated by the patient 12 in the exhalation circuit 30 is greater than or equal to Pe, the air exhaled by the patient 12 can emerge partially toward the outside through the outlet opening 38, and thus, part of the air exhaled by the patient 12 is not usable for phonation.

When Pe is null, phonation is not possible because the valve 40 is open and practically all of the flow of air exhaled by the patient 12 emerges to the outside through the outlet opening 38. This adjustment to a null Pe is generally used in the case where the patient 12 suffers from neuromuscular pathologies, since it makes it possible, with constant mechanical ventilation, to deliver a flow of air to the patient 12 with a relatively low pressure Pi that is therefore better tolerated by the patient 12.

When the patient 12 wishes to speak, he actuates the switch 58, which allows the control means M′ to automatically adjust the pressure supplied by the pressure generator 60 to a value Pe' greater than Pe. The value of Pe' is predefined optimally so as to keep the outlet opening 38 hermetic during the exhalation phase and to thereby ensure the best phonation possible. If Pe is null, the phonation that was not possible during the exhalation phase becomes possible.

Thus, during the exhalation phase and when he actuates the switch 58, the patient 12 can generate an exhaled flow of air with a higher exhalation pressure than when he does not actuate the switch 58, with a maximum threshold corresponding to the pressure Pe′ supplied by the pressure generator 60, without creating leaks toward the outlet opening 38. The patient 12 can then speak with greater intensity.

A fifth embodiment of the invention is shown in FIG. 13 and differs from the fourth embodiment of FIG. 12 in that the pressure generator 60 and the bypass means of the turbine 84 are eliminated.

During the inhalation phase, the turbine 84 delivers a flow of air to the patient 12 with a pressure Pi at the outlet of the turbine 84, while the means M′ automatically control the solenoid valve 80 so as to connect the turbine 84 to the valve 40 and thereby cover the outlet opening 38 with a pressure corresponding to Pi.

During the exhalation phase, the turbine 84 is still connected to the valve 40 and delivers, both to the patient 12 and the valve 40, a pressure Pe lower than or equal to the pressure Pi. In fact, unlike the embodiment of FIG. 12, the inhalation circuit 28 is continuously powered and the turbine 84 can maintain a minimum pressure in the inhalation 28 and exhalation 30 circuits at a value corresponding to the pressure Pe in the valve 40, to within the pressure losses in the inhalation and exhalation circuits 28, 30.

If, during the exhalation phase, the patient 12 does not add any overpressure to the pressure generated by the turbine 84, overpressure that would make it possible to have an exhalation pressure greater than the pressure Pe in the valve 40, then the air produced both by the turbine 84 and the patient 12 during the exhalation phase escapes solely between the trachea 22 and the cannula 26 and can therefore be completely used for phonation.

If the patient 12, while exhaling, makes it possible to have an exhalation pressure greater than Pe, part of the air exhaled by the patient 12 then emerges toward the outside through an outlet opening 38 and is not used for phonation.

If the pressure Pe adjusted by a prescriber on the turbine 84 is null, phonation is not possible because the valve 40 is open and practically all of the flow of air exhaled by the patient 12 emerges to the outside through the outlet opening 38. This adjustment to a null Pe is generally used in the case where the patient 12 suffers from neuromuscular pathologies as it makes it possible, with constant mechanical ventilation, to deliver a flow of air to the patient 12 with a relatively low pressure Pi that is therefore better tolerated by the patient 12.

When the patient 12 wishes to speak, he actuates the switch 58, which allows the control means M′ to adjust the pressure applied by the turbine 84 to the valve 40 to a value Pe' greater than Pe. The value of Pe′ is predetermined optimally, at most equal to Pi, so as to keep the outlet opening 38 hermetic during the exhalation phase and to thereby ensure the best possible phonation. In this way, the patient 12 can speak.

The invention therefore proposes a simple device that makes it possible to facilitate the respiration and speech upon exhalation of a ventilated tracheotomy patient by increasing the pressure level necessary to open the exhalation circuit selectively using a switch, thereby making it possible to increase the intensity of the voice.

In fact, the device forms both an inhalation/exhalation circuit when the patient does not wish to speak, and only an inhalation circuit to restore phonation, preventing or at least reducing the dehydration of his respiratory paths.

The patient can therefore simply and easily, without assistance from a third party, go from an exhalation situation through the upper respiratory paths when he wishes to speak, to an exhalation situation through the tracheotomy cannula when he does not wish to speak.

The device according to the invention thus gives the patient more autonomy, which is an advantage in particular for ventilated tracheotomy patients at home.

1.-10. (canceled)

11. A phonation assistance device for a tracheotomy patient, comprising:
an exhalation circuit connected to a tracheotomy cannula inserted in the trachea of the patient, the exhalation circuit including an outlet opening for the passage of the air exhaled by the patient; and
a valve opening/closing the outlet opening, the valve normally assuming, when the patient inhales, a position closing the outlet opening and, when the patient exhales, a position opening the outlet opening,
the device also comprising priority positive control device of the valve, adapted to selectively bring the valve into the closing position when the patient exhales.

12. The device according to claim 11, wherein the priority positive control device comprises a first solenoid valve for controlling the valve, and a control switch for the first solenoid valve.

13. The device according to claim 11, wherein the valve comprises a housing in which the exhalation circuit emerges and comprising an air outlet opening, and a member steered by the pressure housed in the housing and adapted to assume a closed position in which it closes the outlet opening, and an open position in which it opens the outlet opening.

14. The device according to claim 12, wherein the valve comprises a housing in which the exhalation circuit emerges and comprising an air outlet opening, and a member steered by the pressure housed in the housing and adapted to assume a closed position in which it closes the outlet opening, and an open position in which it opens the outlet opening, and the priority positive control device comprises a pressure generator selectively connected, via the first solenoid valve, to the member controlled by the pressure,

15. The device according to claim 14, wherein the pressure generator is a continuous fan turbine.

16. The device according to claim 11, further comprising an inhalation circuit permanently connected to the cannula and comprising an inlet opening allowing the passage of air inhaled by the patient.

17. The device according to claim 16, further comprising a ventilator whereof the discharge is connected to the inhalation circuit.

18. The device according to claim 17, wherein the ventilator comprises a second solenoid valve and is selectively connected to the valve via the second solenoid valve.

19. The device according to claim 112, further comprising a ventilator whereof the discharge is connected to the inhalation circuit, and the first solenoid valve is selectively controlled by the switch and by the ventilator.

20. The device according to claim Ii, further comprising a tracheotomy cannula inserted into the trachea of the patient and connected to the exhalation circuit.
